(19) United States
(12) Patent Application Publication
(10) Pub. No.: US 2009/0325984 A1
(43) Pub. Date: Dec. 31, 2009

(54) USE OF 1,7-DIMETHYLXANTHINE FOR THE MANUFACTURE OF A NON-ANXIOGENIC PSYCHOANALEPTIC DRUG FOR THE TREATMENT OF A NEUROPSYCHIATRIC DISORDER

(75) Inventors: Jean Costentin, Rouen (FR); Lucilla Mansuy, L'Union (FR); Pierre Sokoloff, Belleserre (FR)

Correspondence Address: BIRCH STEWART KOLASCH & BIRCH, PO BOX 747, FALLS CHURCH, VA, 22040-0747, US

(73) Assignees: Pierre Fabre Medicament, Bulogne-Billancourt (FR); Universite de Rouen, Mont Saint Aignan Cedex (FR)

(21) Appl. No.: 12/304,377

(22) PCT Filed: Jun. 8, 2007
(86) PCT No.: PCT/EP2007/055668

§ 371 Date: Dec. 11, 2008

(30) Foreign Application Priority Data
Jun. 12, 2006 (FR) 0605189

(51) Int. Cl. A61K 31/522; A61P 25/00

(52) U.S. Cl. 514/263.34

(57) ABSTRACT

The present invention relates to the use of paraxanthine for the manufacture of a non-anxiogenic psychoanaleptic drug for the treatment of a neuropsychiatric disorder.

The invention relates to 1,7-dimethylxanthine (1,7-dimethyl-3,7-dihydro-1H-purine-2,6-dione), also known as paraxanthine. Paraxanthine is a natural product known to be present in the plant Sinomenium acutum (Jiang et al., 1998a).

Other methylxanthines are well-known natural products. 1,3,7-Trimethylxanthine (caffeine) is extracted from the beans of Coffea arabica or Coffea robusta. 1,3-Dimethylxanthine (theophylline) is notably present in the leaves of Theacea plants such as Camellia sinensis. 3,7-Dimethylxanthine (theobromine) is notably present in the beans of Theobroma cocoa. These natural methylxanthines are components of beverages or dishes containing coffee, chocolate or tea. In mammals, including man, paraxanthine is also a caffeine metabolite (Yesair et al., 1984).

Caffeine is classified as a psychostimulant, as are cocaine, amphetamine, methamphetamine and methylphenidate. Current caffeine-based beverages and products for human consumption are well-known for their properties of stimulating alertness, concentration, attention and intellectual functions. Other psychostimulants, such as methylphenidate, are used as therapeutic agents to treat the pathology known as attention-deficit/hyperactivity disorder (ADHD).

Caffeine is also reputed to induce anxiety states and can sometimes cause panic attacks. For example, patients who consume large quantities of coffee may suffer from generalized anxiety symptoms referred to as “caffeinism” (Greden, 1974). Experimentally, the administration of high doses of caffeine produces increases in anxiety measurements in healthy volunteers (Stern et al., 1989). The anxiogenic effects of caffeine are more intense in patients prone to panic attacks (Boulenger et al., 1984). A panic attack, according to DSM-III-R criteria (American Psychiatric Association, 1987), can be caused experimentally by administering caffeine (Nickel and Uhde, 1994). Lastly, in another experimental study with adolescents, the subjects stated that caffeine made them anxious (Bernstein et al., 1994).

Surprisingly, the inventors have discovered that paraxanthine, in contrast to caffeine, loses its anxiogenic activity in animals, and, in addition, has anxiolytic activity. Thus, the inventors propose the use of paraxanthine for the manufacture of a non-anxiogenic psychoanaleptic drug to treat neuropsychiatric disorders for which sleep disorders and anxiety disorders are among the symptoms.

According to the classification suggested by Delay and Denicker (1957) and validated during the World Congress of Psychiatry in 1961, “psychoanaleptic drug,” from the Greek psyche, meaning the mind and analeptikos, meaning restorative, means a pharmacological agent that induces alertness, reduces the desire to doze off and stimulates thought, attention and intellectual faculties. Paraxanthine is not classified as a psychoanaleptic substance in current pharmacology texts. In animals, notably rodents, psychoanaleptic effects are evaluated by measuring locomotor activity when the animal is placed in a novel environment.

“Anxiety” means feelings of imminent and unspecified danger accompanied by general apprehension, helplessness or fear. “Anxiogenic” means any effect likely to create anxiety or to increase measurements of anxiety. An anxiogenic situation can be created in animals, particularly rodents, by placing them in unusual situations which appear to them to be dangerous. “Anxiolytic” means any effect that opposes anxiety or an increase in anxiety. In an animal, an anxiolytic effect is demonstrated when the animal loses its apprehension of a situation which it senses as dangerous and moves further into an environment related to this situation or spends more time in this situation.

Idiopathic hypersomnia is a disorder that combines extended nocturnal sleep, difficulty waking, often with confusion, and more or less permanent daytime sleepiness.

Narcolepsy is a disorder characterized by excessive daytime sleepiness, expressed by irrepressible fits of sleep that occur several times per day and last from two to 30 minutes. These fits of sleep are followed by normal alertness, but only for a few hours. These continual fluctuations of alertness are accompanied by attention and memory difficulties.

Depression is a common mood disorder characterized by feelings of intense sadness, pessimistic anxiety and self depreciation, often accompanied by a loss of enthusiasm, energy or drive, fatigue, anhedonia or difficulty experiencing pleasure, and sleep disorders. The diagnosis of major depression, or a major depressive episode, is made when the patient exhibits the depression criteria described in detail in the DSM-III-R (American Psychiatric Association, 1994). Less severe forms are regarded as uncharacterized depressive disorders or dysthymia, and can persist for several years. Depressed patients are treated with antidepressants, which often have side effects that are difficult to deal with, such as anxiety, somnolence and fatigue.

Functional disorders are those that relate to broad physiological functions and that are not due to organic lesions but rather to the manner in which an organ, such as the liver or heart, functions. Functional disorders can be the cause of an illness that arises at a later date.

Attention-deficit/hyperactivity disorder, or ADHD, is the most common childhood neuropsychiatric disorder. ADHD is characterized by three primary symptoms: inattention, hyperactivity and impulsiveness. All three of these symptoms can be present in children with ADHD, but to differing degrees. Consequently, the disorder is subdivided into three types: combined, predominantly inattentive and predominantly hyperactive/impulsive (American Psychiatric Association, 1987).

“Pharmaceutically acceptable” refers to molecular entities and compositions that do not produce any adverse effects, allergic effects or other undesirable reactions when administered in animals or man.

When used here, the term “pharmaceutically acceptable excipient” includes any diluent, adjuvant or excipient, such as preservatives, fillers, disintegrants, wetting agents, emulsifiers, dispersants, antibacterials, antifungals or agents that delay intestinal and digestive resorption. The use of these media or vectors is well-known in the art. Unless the agent is chemically incompatible with paraxanthine, its use in therapeutic compositions with paraxanthine may be considered. Other therapeutic agents may also be incorporated in therapeutic compositions containing paraxanthine.

In the context of the invention, the term treatment, as used here, means preventing or inhibiting the occurrence or progression of the disorder to which the term is applied, or of one or more of the symptoms thereof.

“Therapeutically-active quantity” means a quantity of paraxanthine that is effective in obtaining the desired therapeutic effect according to the invention.

According to the invention, the term “patient” refers to a human or to a non-human mammal affected or potentially affected by a given pathology. Preferentially, the patient is human.

The inventors have demonstrated that in the mouse, paraxanthine exerts a dose-dependant stimulating effect on locomotor activity at doses from 1 mg/kg up to 50 mg/kg (see Example 1). Under the same conditions, caffeine also exerts a stimulating effect, but the effect is less and it occurs in a narrower range of doses (10-25 mg/kg).

In the hole-board test, which measures the anxiogenic or anxiolytic capacity of a substance, caffeine, at a dose of 50 mg/kg, has an anxiogenic effect, which is not the case with paraxanthine at a dose of 50 mg/kg (see Example 2).

In the black-white box test, which also measures the anxiogenic or anxiolytic capacity of a substance, paraxanthine at a dose of 50 mg/kg has an anxiolytic effect, whereas caffeine, at the same dose of 50 mg/kg, does not (see Example 3).

In the raised cross-shaped labyrinth test, which measures the anxiogenic or anxiolytic capacity of a substance, caffeine (at a dose of 50 mg/kg), but not paraxanthine (at a dose of 50 mg/kg) is anxiogenic. Paraxanthine, at a dose of 50 mg/kg, has an anxiolytic effect (see Example 4).

In the Vogel conflict test, which measures the anxiogenic or anxiolytic capacity of a substance in a conflict situation in which a thirsty rat receives punishment in the form of a mild electric shock each time it consumes water, paraxanthine did not have an anxiogenic effect (see Example 5).

In the four-plate test, in which a mouse is punished by a mild electrical shock when crossing between two plates, which is a normal exploration behavior in this species, anxiolytic substances increase the number of crossings between plates. In this test, paraxanthine has anxiolytic activity at a dose of 25 mg/kg (see Example 6).

Thus, paraxanthine demonstrates in animals a stimulating effect that is at least as great as that of caffeine, while remaining non-anxiogenic, and in certain tests even shows anxiolytic effects. According to the most current understanding of those persons skilled in the art, no pharmacological agent is known to have psychoanaleptic drug activity without being anxiogenic. No product is known in the current state of the art that combines psychoanaleptic and anxiolytic properties. Similarly, all known anxiolytic agents, particularly minor tranquilizers of benzodiazepine structure, induce sleep.

The inventors thus propose the use of paraxanthine in therapeutic compositions for the treatment of sleep disorders or anxiety disorders, for the disorders listed here as examples, without being limited to these examples in any way.

In a first aspect of the invention, paraxanthine is used for the treatment of idiopathic hypersomnia and narcolepsy. Hypersomnia is the primary symptom of the latter disorder and paraxanthine can relieve such patients without causing anxiety or increasing anxiety.

In another aspect of the invention, paraxanthine is used to treat patients suffering from depression. Fatigue, psychomotor slowing and sleep disorders are symptoms of depression, and are often associated with anxiety. According to the invention, paraxanthine can be used to treat patients suffering from major depression, uncharacterized depressive disorders or dysthymia. Preferentially, these patients suffer from sleep disorders, accompanied or not by anxiety.

Another aspect of the invention involves treating attention-deficit/hyperactivity disorder with paraxanthine. This disorder is currently treated with psychostimulants such as methylphenidate. According to the invention, paraxanthine will have a beneficial effect on attention-deficit/hyperactivity disorder by its non-anxiogenic psychoanaleptic effect which increases concentration and stimulates intellectual faculties.

Another aspect of the invention involves treating with paraxanthine patients who suffer from functional disorders. These disorders are often associated with psychomotor slowing and fatigue, symptoms which could be improved by paraxanthine, without causing anxiety, a factor which aggravates these disorders. The invention is not limited to the disorders mentioned above and may be of use for chronic fatigue, irritable bowel syndrome and fibromyalgia, among others.

According to a specific characteristic of the invention, paraxanthine is used to manufacture a non-anxiogenic psychoanaleptic drug for the treatment of fatigue and sleep or concentration disorders associated with depression, fibromyalgia, irritable bowel syndrome, nicotine withdrawal, Parkinson's disease, multiple sclerosis, amyotrophic lateral sclerosis, jet lag or shift work.

According to another specific characteristic of the invention, paraxanthine is used to manufacture a non-anxiogenic psychoanaleptic drug for the treatment of anxiety disorders associated with depression or nicotine withdrawal.

Considering that the absence of anxiogenic effects, and even the induction of anxiolytic effects, associated with the stimulating effects of paraxanthine can have only favorable effects on attention and memory, the inventors also propose the use of paraxanthine to treat cognitive deficits, for example the mild or moderate cognitive deficits related to aging, which are often an early form of dementia or Alzheimer's disease. Cognitive disorders also accompany psychiatric disorders such as schizophrenia. According to the invention, paraxanthine can be used as an adjuvant in the treatment of schizophrenia or of other forms of psychoses.

Considering that fatigue and sleep disorders can accompany neurological disorders, paraxanthine can be used as an adjuvant in the treatment of these disorders. The invention is not limited to these disorders, and may be of use for multiple sclerosis, Parkinson's disease and amyotrophic lateral sclerosis, among others.

Paraxanthine can be used according to the invention in pharmaceutically acceptable preparations for the treatment of various diseases or disorders, in particular those whose symptoms include sleep disorders and anxiety.

Paraxanthine is prepared by chemical synthesis according to methods known in the art. One example that can be mentioned is the total synthesis of paraxanthine from isopropylhydrazine and 2-cyano-3-ethoxy-acrylic acid ethyl ester by Schmidt and colleagues (Schmidt et al., 1958). Other synthetic routes can be used to obtain paraxanthine, for example starting with xanthine (Müller et al., 1993).

Paraxanthine can also be prepared from the extracts of plants or organisms that synthesize it. One of these known plants is Sinomenium acutum (Jiang et al., 1998b), however the invention is not limited to the use of this plant alone for the extraction of paraxanthine.

Paraxanthine can also be obtained by the selective demethylation of caffeine via a biochemical route. Caffeine is incubated with an enzymatic preparation containing CYP1A2 activity, or CYP1A2-analog activity, of human or non-human origin, for example extracted from tissue such as the liver, which catabolizes in mammals the selective conversion of caffeine into paraxanthine.

Paraxanthine can also be obtained by using a microorganism that already exists in nature or one that is genetically modified. For example, a microorganism can be used into which the gene coding for the CYP1A2 enzyme of human or non-human origin has been introduced. The introduction of a foreign gene into a microorganism by a plasmid or viral vector is well-known in the art.

The use according to the invention involves paraxanthine regardless of the method by which it is obtained, for example by chemical synthesis or from a plant extract. A pharmaceutical composition according to the invention contains paraxanthine in a therapeutically-active quantity. The quantity of paraxanthine required is such that the dose administered is between 0.1 mg and 100 mg per kg of body weight per day, preferably between 0.5 mg and 20 mg per kg of body weight per day. Another pharmaceutical composition comprises a combination of paraxanthine in a therapeutically-active quantity and a pharmaceutically-acceptable excipient.

Another pharmaceutical composition according to the invention contains paraxanthine in a therapeutically-active quantity and another active ingredient used to treat a psychiatric or neurological disorder. This other active ingredient can be an antidepressant, an anxiolytic, an antipsychotic, an antiparkinsonian, an acetylcholine esterase inhibitor, an anti-inflammatory, in particular a corticoid, memantine or riluzole.

Paraxanthine can be administered by oral, parenteral, rectal or nasal routes. In particular, paraxanthine can be administered by oral route in a suitable formulation. A formulation suitable for administration to a patient by oral route is a therapeutic unit such as a gelatin capsule, a tablet, a powder, granules, a solution, a suspension in an aqueous or non-aqueous liquid, or an oil/water liquid emulsion. Each formulation contains a dose of paraxanthine predetermined to be therapeutically active.

Various effects exerted by paraxanthine have been objectified in the examples mentioned below and summarized in the attached figures as follows:

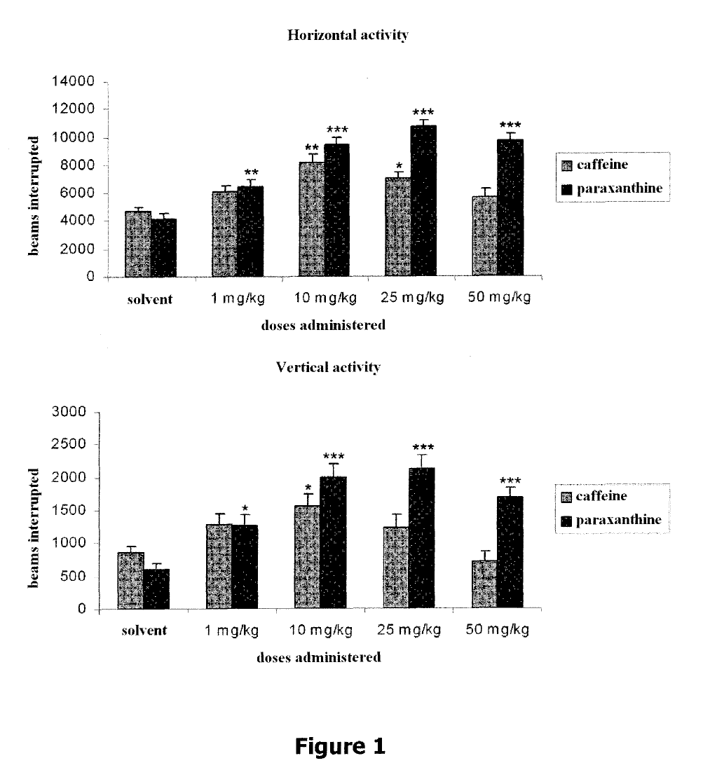

FIG. 1: Comparative effects of paraxanthine and caffeine on locomotor activity (horizontal in top graph, vertical in bottom graph) over a period of 60 minutes in the mouse. The results are given as mean±SEM (n=14 animals per group); *P<0.05, **P<0.01 and ***P<0.001 vs. control animals treated with solvent.

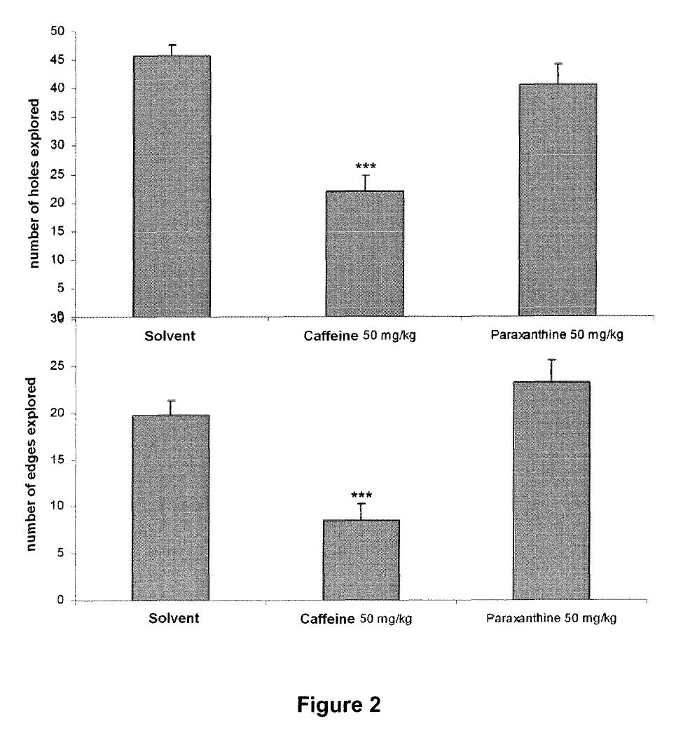

FIG. 2: Anxiogenic effect of caffeine, but not paraxanthine, in the mouse in the hole-board test (number of holes explored in top graph, number of edges explored in bottom graph). The results are given as mean±SEM (n=8 animals per group); ***P<0.001 vs. control animals receiving solvent.

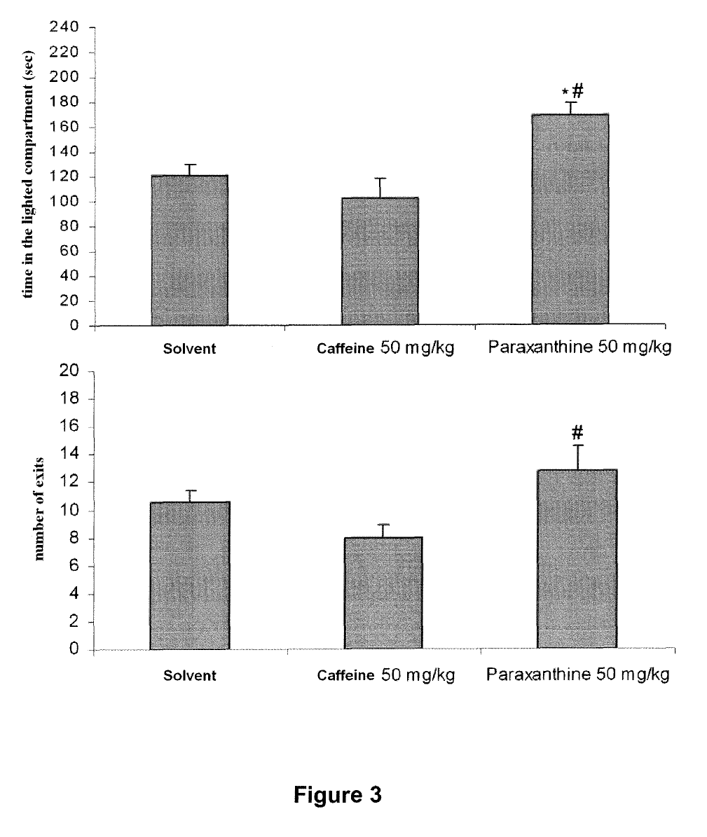

FIG. 3: Anxiolytic effect of paraxanthine, but not caffeine, in the mouse in the black-white box test (time spent in the white compartment in the top graph, number of entries into the white compartment in the bottom graph). The results are given as mean±SEM (n=10 animals per group); *P<0.05 vs. control animals receiving solvent; #P<0.01 vs. animals receiving caffeine.

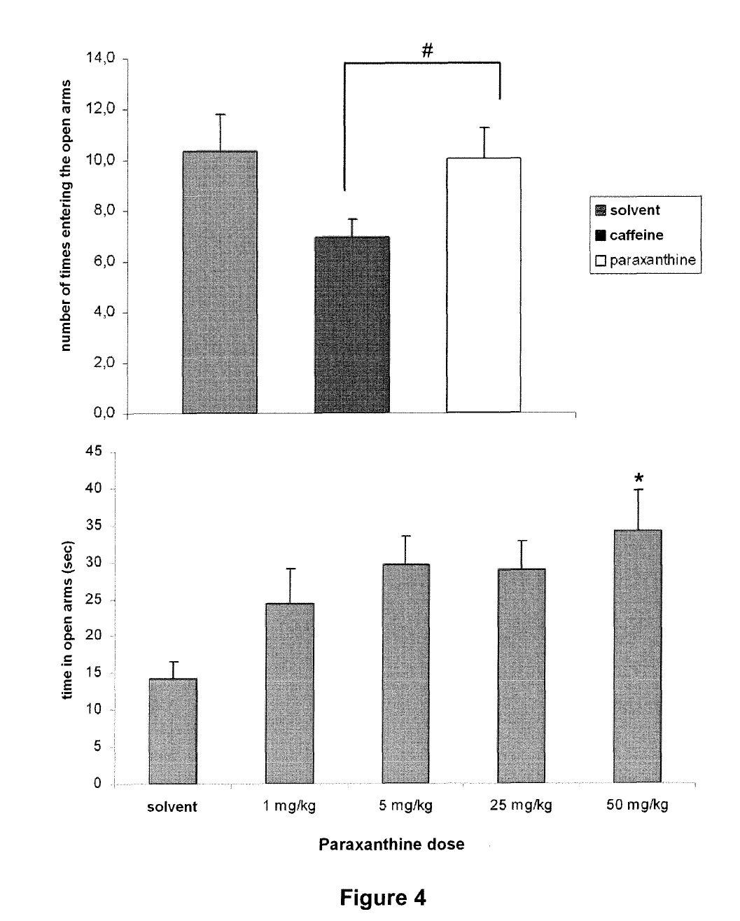

FIG. 4: Anxiogenic effect of caffeine and anxiolytic effect of paraxanthine in the mouse in the raised labyrinth test. Caffeine reduces the number of times entering into the open arm, compared to paraxanthine (top graph). Paraxanthine increases the time spent in the open arm (bottom graph). The results are given as mean±SEM (n=15-20 animals per group); *P<0.05 vs. control animals receiving solvent; #P<0.01 vs. animals receiving caffeine.

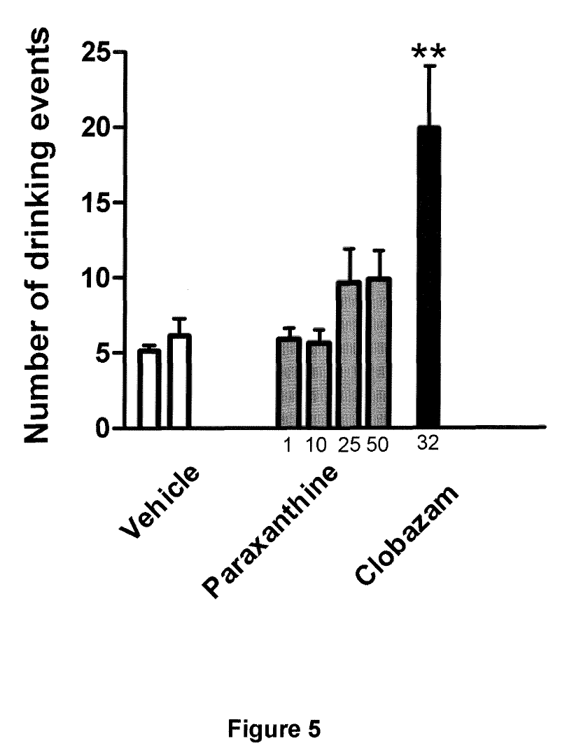

FIG. 5: Absence of anxiogenic effect of paraxanthine in the Vogel conflict test in the rat. The results are given a mean±SEM (n=10 animals) and represent the number of times water was consumed. Clobazam is used as the reference anxiolytic. The doses of paraxanthine and clobazam are indicated in mg/kg below the columns. The first “vehicle” column corresponds to the solvent used for paraxanthine; the second “vehicle” column corresponds to the solvent used for clobazam; **P<0.01 vs. the respective solvent.

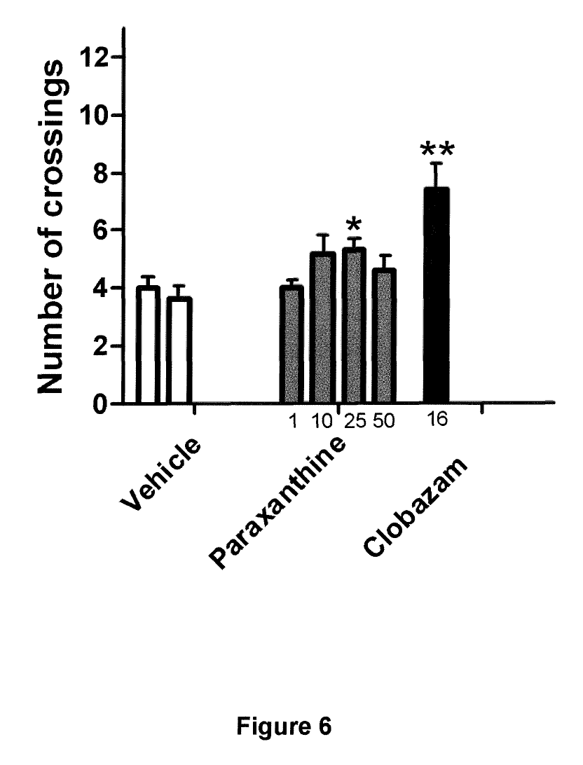

FIG. 6: Anxiolytic effect of paraxanthine in the four-plate test. The results are given as mean±SEM (n=10 animals) and represent the number of crossings between two plates. Clobazam is used as the reference anxiolytic. The first “vehicle” column corresponds to the solvent used for paraxanthine; the second “vehicle” column corresponds to the solvent used for clobazam; *P<0.05 and **P<0.01 vs. the respective solvent.

The invention will be better understood upon consideration of the examples below:

### EXAMPLE 1

### Stimulating Effect of Paraxanthine in the Mouse, Measured by Locomotor Activity

Experiments were performed with male CD1 albino mice (Charles River) weighing 25 to 35 grams at the time of the experiment. The animals were placed in groups of 20 in plexiglass cages (38×24×18 cm) and kept in a ventilated animal facility where the temperature was maintained at 21±1° C. The animals had free access to water and food; artificial lighting established a day/night cycle (daytime between 7:00 a.m. and 7:00 p.m.). Experiments were conducted between 11:00 a.m. and 6:00 p.m.

Caffeine and paraxanthine were acquired from Sigma; they were dissolved under heating in a sodium benzoate solution (Sigma) to a concentration of 30 mg/ml. The solutions were stabilized with Cremophor EL (Sigma) to a final concentration of 15%. The solutions were injected by intraperitoneal route in a dose of 10 ml/kg.

Before each test, the animals were isolated for 20 minutes in plexiglass cages (27×13×13 cm); they had food at their disposal. For locomotor activity measurements, a computerized activity monitoring system was used comprised of individual plexiglass chambers (20 cm on each side and 30 cm in height) with a plexiglass cover and floor. Photoelectric sensors in the chambers measured the horizontal and vertical activity of the animals, expressed as the number of interrupted beams, and the data was analyzed using a software application (Omnitech Electronics Inc., Columbus, Ohio, USA). Animal locomotor activity was measured for six consecutive 10-minute periods; the room in which measurements were taken was dark. Animals were placed in the activity monitoring system immediately after receiving the injection. The chambers were cleaned after each animal's test.

Paraxanthine stimulated horizontal locomotor activity starting with a dose of 1 mg/kg, from the first measurement period and for 30 minutes thereafter (P<0.05 or P<0.01). At higher doses, the effect was more marked (P<0.01 or P<0.001) and more long-lasting (at least one hour). These results are confirmed by analysis of cumulative horizontal activity (FIG. 1, top graph). In similar experiments, caffeine, starting with a dose of 10 mg/kg, stimulated animals' horizontal locomotor activity within the first 10 minutes of the experiment; this action lasted for at least one hour (P<0.05). At a dose of 25 mg/kg, caffeine stimulated this activity 10 minutes after the injection, an effect which lasted 40 minutes (P<0.05). For doses lower than 10 mg/kg or higher than 25 mg/kg, no significant difference was demonstrated compared to animals having received solvent alone. It should be noted that horizontal activity for one hour under experimental conditions is stimulated by caffeine only at doses of 10 mg/kg (P<0.01) and 25 mg/kg (P<0.05) (FIG. 1).

Paraxanthine also stimulated the animals' vertical locomotor activity, in a way comparable to that of horizontal activity, although the effect of the 1 mg/kg dose is more difficult to demonstrate (FIG. 1, bottom graph). Caffeine also stimulated the animals' vertical locomotor activity at the 10 mg/kg dose, but this effect is only statistically significant 30 minutes after the injection (P<0.05). For the 100 mg/kg dose, the animals' vertical activity decreased relative to that of the controls from the first measurement period and for 40 minutes thereafter (P<0.05). A similar pattern is observed when cumulative vertical activity is considered (FIG. 1, bottom graph).

### EXAMPLE 2

### Non-Anxiogenic Effect of Paraxanthine in the Mouse as Measured by the Hole-Board Test

Experiments were performed on mice of the same strain maintained under the same conditions as in Example 1.

The hole-board test investigates curiosity and is negatively affected by anxiety. This apparatus, placed 60 cm above the floor, consists of a square platform of opaque plastic, 40 cm on each side, with 16 evenly distributed holes of such size that an animal is able to pass its head through. The animals were injected and then isolated for 20 minutes before being placed at the center of the platform. The number of holes and edges explored by each animal was counted. The apparatus was cleaned after each animal's test.

Caffeine, at a dose of 50 mg/kg, significantly decreased the number of holes (FIG. 2, top graph) and edges (FIG. 2, bottom graph) explored by the animals (P<0.001) whereas paraxanthine at the same dose did not have any effect in this respect. In this test, therefore, caffeine had an anxiogenic effect whereas paraxanthine did not.

### EXAMPLE 3

### Anxiolytic Effect of Paraxanthine, But Not of Caffeine, in the Mouse as Measured by the Black-White Box Test

Experiments were performed on mice of the same strain maintained under the same conditions as in Example 1.

The black-white box test measures the state of anxiety of animals as a function of their aversion to light. The apparatus consists of two compartments of the same size (length=21 cm, width=15 cm, height=25 cm), one painted white and illuminated by a 40 W bulb, the other painted black and closed with a cover. The animal can pass from one compartment to the other via to an opening 5 cm-square in the lower portion of the partition. After the injection, the animals were isolated for 20 minutes and then placed in the black compartment with their head facing the corner opposite the opening. Using a mirror placed above the apparatus, the time before first entering the lit compartment, the number of times entering and the time spent in the lighted compartment were measured. The two compartments were cleaned between each test.

Caffeine, at a dose of 50 mg/kg, did not change the amount of time spent in the lighted compartment and thus did not have an anxiolytic effect. On the other hand, paraxanthine increased the amount of time spent in the lighted compartment compared to controls (P<0.05) and compared to the “caffeine” group (P<0.01) (FIG. 3, top graph). Moreover, animals treated with paraxanthine went into the lighted compartment more often than animals treated with caffeine (P<0.05) (FIG. 3, bottom graph). Lastly, neither of the two products tested influenced the amount of time before first leaving the black compartment. Thus, in this test, paraxanthine exhibited an anxiolytic effect.

### EXAMPLE 4

### Anxiolytic Effect of Paraxanthine and Anxiogenic Effect of Caffeine in the Mouse as Measured by the Raised Cross-Shaped Labyrinth Test

Experiments were performed on mice of the same strain maintained under the same conditions as in Example 1.

The raised cross-shaped labyrinth test measures the animals' anxiety level based on its spontaneous aversion to voids (80). The apparatus is composed of four arms, placed at right angles, each measuring 18×6 cm; it rests on a pedestal 60 cm above the floor. Two of the arms have 6 cm-high side walls and are laid out end to end; these are the “closed” arms. The other two arms, at right angles to the closed arms, do not have side walls; these are the “open” arms. After receiving their injections, the animals were isolated for twenty minutes and then placed in the labyrinth, at the center of the cross, with their head in the direction of a closed arm. Movements by the animals were recorded for five minutes by a video camera connected to image analysis software (Videotrack). The labyrinth was cleaned after each animal's test.

Caffeine at a dose of 50 mg/kg reduced the number of times the mice entered the open arm (P<0.05), whereas paraxanthine had no effect (FIG. 4, top graph). This suggests that caffeine has an anxiogenic effect, which is not the case with paraxanthine.

Paraxanthine was tested at increasing doses (from 1 mg/kg to 50 mg/kg) and the time spent by the animals in the open arm increased until a significant effect was exhibited at a dose of 50 mg/kg (P<0.05, FIG. 4, bottom graph). Thus it appears that at high doses paraxanthine has an anxiolytic effect in the raised cross-shaped labyrinth test.

### EXAMPLE 5

### Absence of Anxiogenic Effect of Paraxanthine in the Rat as Measured by the Vogel Conflict Test

Experiments were performed using male Wistar rats weighing 180 g to 280 g according to the procedure described by Vogel et al. (Psychopharmacologia, 1971, 21: 1-7). Animals were deprived of water for 48 hours and then individually placed in a plexiglass chamber (15×32×34 cm) whose floor consisted of conducting metal bars spaced 1 cm apart. In the center of one of the chamber's walls was placed a metal cup connected to an electric shock generator (1.7 mA; 1 s).

During the test, the animal, which is left free to explore the apparatus, receives an electric shock each time it consumes water from the cup. A researcher blind to the treatment received by the animal counts the number of times that the animal consumes water and is punished by the electric shock. An increase in the number of times an animal consumes water indicates an anxiolytic effect, whereas a reduction in this number indicates an anxiogenic effect.

Paraxanthine was dissolved under heating in sodium benzoate (30 mg/ml) with the addition of Cremophor EL to a final concentration of 15% and administered by intraperitoneal route at doses of 1 mg/kg, 10 mg/kg, 25 mg/kg and 50 mg/kg. Clobazam was used as the reference anxiolytic; it was dispersed in a 0.2% hydroxypropylmethylcellulose solution and administered by intraperitoneal route at a dose of 32 mg/kg.

Paraxanthine did not decrease the number of times animals consumed water and thus did not demonstrate an anxiogenic effect (FIG. 5).

### EXAMPLE 6

### Anxiolytic Effect of Paraxanthine in the Mouse as Measured by the Four-Plate Test

Experiments were performed using NMRI mice weighing 20 g to 30 g according to the method described by Aron et al. (Neuropharmacology, 1971, 10:459-469). Animals were placed in a plastic chamber whose floor consisted of four metal plates independently connected to an electric shock generator (2.5 mA; 1.5 s). The animal is initially left free to explore the apparatus for 15 seconds, after which it receives an electric shock each time it crosses between two metal plates. An increase in the number of crossings indicates anxiolytic activity, whereas a reduction in this number indicates anxiogenic activity.

Paraxanthine was dissolved under heating in sodium benzoate (30 mg/ml) with the addition of Cremophor EL to a final concentration of 15% and administered by intraperitoneal route at doses of 1 mg/kg, 10 mg/kg, 25 mg/kg and 50 mg/kg. Clobazam was used as the reference anxiolytic; it was dispersed in a 0.2% hydroxypropylmethylcellulose solution and administered by intraperitoneal route at a dose of 16 mg/kg.

Paraxanthine exhibited anxiolytic activity at a dose of 25 mg/kg (P<0.05) (FIG. 6).

### REFERENCES

American Psychiatric Association (1987) Diagnostic and statistical manual of mental disorders. Washington, D.C.: American Psychiatric Association.
American Psychiatric Association (1994) Diagnostic and statistical manual of mental disorders—Fourth Edition (DSM-IV).
Bernstein G A, Carroll M E, Crosby R D, Perwien A R, Go F S, Benowitz N L (1994) Caffeine effects on learning, performance, and anxiety in normal school-age children. J Am Acad Child Adolesc Psychiatry 33: 407-415.
Boulenger J P, Uhde T W, Wolff E A, III, Post R M (1984) Increased sensitivity to caffeine in patients with panic disorders. Preliminary evidence. Arch Gen Psychiatry 41: 1067-1071.
Greden J F (1974) Anxiety or caffeinism: a diagnostic dilemma. Am J Psychiatry 131: 1089-1092.
Jiang M, Kameda K, Han L K, Kimura Y, Okuda H (1998) Isolation of lipolytic substances caffeine and 1,7-dimethylxanthine from the stem and rhizome of Sinomenium actum. Planta Med 64: 375-377.
Müller C E, Shi D, Manning M Jr, Daly J W (1993) Synthesis of paraxanthine analogs (1,7-disubstituted Xanthines) and other xanthines unsubstituted at the 3-position: structure-activity relationships at adenosine receptors. J Med Chem 36: 3341-3349.
Nickell P V, Uhde T W (1994) Dose-response effects of intravenous caffeine in normal volunteers. Anxiety 1: 161-168.
Schmidt P, Eichenberger K, Druey J (1958) 1-Pyrazolo[3,4-d]pyrimidines with caffeine-like structure and activity. Helv Chim Acta 41: 1052-1060.
Stern K N, Chait L D, Johanson C E (1989) Reinforcing and subjective effects of caffeine in normal human volunteers. Psychopharmacology (Berl) 98: 81-88.
Yesair D W, Branfman A R, Callahan M M (1984) Human disposition and some biochemical aspects of methylxanthines. Prog Clin Biol Res 158: 215-233.

1. The use of paraxanthine for the manufacture of a non-anxiogenic psychoanaleptic drug for the treatment of a neuropsychiatric disorder.

2. The use according to claim 1, wherein the neuropsychiatric disorder is fatigue, a sleep disorder or an attention disorder.

3. The use according to claim 1, wherein the neuropsychiatric disorder is an anxiety disorder.

4. The use according to claim 1, wherein the neuropsychiatric disorder is a cognitive disorder.

5. The use according to claim 2, wherein the sleep disorder or attention disorder is idiopathic hypersomnia or narcolepsy.

6. The use according to claim 2, wherein the drug is for treating the fatigue, sleep disorder or attention disorder of a patient affected by depression, fibromyalgia, irritable bowel syndrome, nicotine withdrawal, Parkinson's disease, multiple sclerosis, amyotrophic lateral sclerosis, jet lag or shift work.

7. The use according to claim 3, wherein the drug is for treating the anxiety disorder of a patient affected by depression or by nicotine withdrawal.

8. The use according to claim 1, wherein the drug is for treating attention-deficit/hyperactivity disorder.

9. The use according to claim 4, wherein the drug is for treating the cognitive disorder of a patient suffering from schizophrenia.

10. The use according to claim 4, wherein the cognitive disorder is related to aging or to Alzheimer's disease.
